(19) United States
(12) Patent Application Publication
(10) Pub. No.: US 2024/0081744 A1
(43) Pub. Date: Mar. 14, 2024

(54) EMBEDDED SENSOR IMPLANT DEVICES

(71) Applicant: EDWARDS LIFESCIENCES CORPORATION, Irvine, CA (US)

(72) Inventors: Kokou Anani Amefia, Aliso Viejo, CA (US); Rani Abdullah Mahmoudi, Huntington Beach, CA (US)

(21) Appl. No.: 18/513,231

(22) Filed: Nov. 17, 2023

Related U.S. Application Data
(63) Continuation of application No. PCT/US2022/030198, filed on May 20, 2022.
(60) Provisional application No. 63/225,689, filed on Jul. 26, 2021.
(60) Provisional application No. 63/225,039, filed on Jul. 23, 2021.
(60) Provisional application No. 63/224,286, filed on Jul. 21, 2021.
(60) Provisional application No. 63/191,534, filed on May 21, 2021.

(51) Int. Cl. A61B 5/00; A61B 90/00

(52) U.S. Cl. CPC A61B 5/6882; A61B 5/6852; A61B 90/08; A61B 2560/0462; A61B 2560/066; A61B 2562/16

(57) ABSTRACT

A sensor implant device comprises a sensor body, at least a first sensor component, and one or more anchoring features coupled to the sensor device and configured to anchor within a tissue wall. The one or more anchoring features are configured to assume an unexpanded form during delivery and configured to expand into the tissue wall.

### RELATED APPLICATIONS

This application is a continuation of International Patent Application No. PCT/US2022/030198, filed May 20, 2022, and entitled EMBEDDED SENSOR IMPLANT DEVICES, which claims priority to U.S. Provisional Patent Application Ser. No. 63/191,534, filed on May 21, 2021 and entitled IMPLANT-COUPLED SENSORS, U.S. Provisional Patent Application Ser. No. 63/224,286, filed on Jul. 21, 2021 and entitled IMPLANT-ADJACENT SENSOR ANCHORING, U.S. Provisional Patent Application Ser. No. 63/225,039, filed on Jul. 23, 2021 and entitled SHUNT BARREL SENSOR IMPLANT ANCHORING, U.S. Provisional Patent Application Ser. No. 63/225,689, filed on Jul. 26, 2021 and entitled EMBEDDED SENSOR IMPLANT DEVICES, the complete disclosures of which are hereby incorporated by reference in their entirety.

### BACKGROUND

### Field

The present disclosure generally relates to the field of medical implant devices.

### Description of Related Art

Various medical procedures involve the implantation of medical implant devices within the anatomy of the heart. Certain physiological parameters associated with such anatomy, such as fluid pressure, can have an impact on patient health prospects.

### SUMMARY

Described herein are one or more methods and/or devices to facilitate monitoring of physiological parameter(s) associated with certain chambers and/or vessels of the heart, such as the left atrium, using one or more sensor implant devices.

For purposes of summarizing the disclosure, certain aspects, advantages and novel features have been described. It is to be understood that not necessarily all such advantages may be achieved in accordance with any particular example. Thus, the disclosed examples may be carried out in a manner that achieves or optimizes one advantage or group of advantages as taught herein without necessarily achieving other advantages as may be taught or suggested herein.

### BRIEF DESCRIPTION OF THE DRAWINGS

Various examples are depicted in the accompanying drawings for illustrative purposes and should in no way be interpreted as limiting the scope of the inventions. In addition, various features of different disclosed examples can be combined to form additional examples, which are part of this disclosure. Throughout the drawings, reference numbers may be reused to indicate correspondence between reference elements.

FIG. 1 illustrates an example representation of a human heart in accordance with one or more examples.

FIG. 2 illustrates example pressure waveforms associated with various chambers and vessels of the heart according to one or more examples.

FIG. 3 illustrates a graph showing left atrial pressure ranges.

FIG. 4 is a block diagram representing an implant device in accordance with one or more examples.

FIG. 5 is a block diagram representing a system for monitoring one or more physiological parameters associated with a patient according to one or more examples.

FIG. 6 illustrates an example sensor assembly/device that can be a component of a sensor implant device, in accordance with one or more examples.

FIG. 7 illustrates a sensor implant device comprising a sensor assembly/device and/or one or more anchoring features, in accordance with one or more examples.

FIG. 8A illustrates a collapsed/compressed form of a sensor implant device in accordance with one or more examples.

FIG. 8B illustrates an expanded form of the sensor implant device in accordance with one or more examples.

FIG. 9 illustrates a sensor implant device delivered via a catheter, in accordance with one or more examples.

FIG. 10 illustrates a delivery process for delivering a sensor implant device via a catheter to a tissue wall of a left atrium, in accordance with one or more examples.

FIG. 11 illustrates a delivery process for delivering a sensor implant device via a catheter to a tissue wall of a coronary sinus, in accordance with one or more examples.

FIG. 12 provides a flowchart for an example process for percutaneous delivery and/or use of one or more of the various sensor implant devices described herein in accordance with one or more examples.

### DETAILED DESCRIPTION

The headings provided herein are for convenience only and do not necessarily affect the scope or meaning of the claimed invention.

Although certain preferred examples and examples are disclosed below, inventive subject matter extends beyond the specifically disclosed examples to other alternative examples and/or uses and to modifications and equivalents thereof. Thus, the scope of the claims that may arise herefrom is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, for example, various examples may be carried out in a manner that achieves or optimizes one advantage or group of advantages as taught herein without necessarily achieving other aspects or advantages as may also be taught or suggested herein.

Certain reference numbers are re-used across different figures of the figure set of the present disclosure as a matter of convenience for devices, components, systems, features, and/or modules having features that may be similar in one or more respects. However, with respect to any of the examples disclosed herein, re-use of common reference numbers in the drawings does not necessarily indicate that such features, devices, components, or modules are identical or similar. Rather, one having ordinary skill in the art may be informed by context with respect to the degree to which usage of common reference numbers can imply similarity between referenced subject matter. Use of a particular reference number in the context of the description of a particular figure can be understood to relate to the identified device, component, aspect, feature, module, or system in that particular figure, and not necessarily to any devices, components, aspects, features, modules, or systems identified by the same reference number in another figure. Furthermore, aspects of separate figures identified with common reference numbers can be interpreted to share characteristics or to be entirely independent of one another.

Certain standard anatomical terms of location are used herein to refer to the anatomy of animals, and namely humans, with respect to the preferred examples. Although certain spatially relative terms, such as “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa.

The present disclosure relates to systems, devices, and methods for monitoring of one or more physiological parameters of a patient (e.g., blood pressure) using sensor-integrated cardiac shunts and/or other medical implant devices. In some implementations, the present disclosure relates to cardiac shunts and/or other cardiac implant devices that incorporate or are associated with pressure sensors or other sensor devices. The term “associated with” is used herein according to its broad and ordinary meaning. For example, where a first feature, element, component, device, or member is described as being “associated with” a second feature, element, component, device, or member, such description should be understood as indicating that the first feature, element, component, device, or member is physically coupled, attached, or connected to, integrated with, embedded at least partially within, or otherwise physically related to the second feature, element, component, device, or member, whether directly or indirectly. Certain examples are disclosed herein in the context of cardiac implant devices. However, although certain principles disclosed herein are particularly applicable to the anatomy of the heart, it should be understood that sensor implant devices in accordance with the present disclosure may be implanted in, or configured for implantation in, any suitable or desirable anatomy.

Cardiac Physiology

The anatomy of the heart is described below to assist in the understanding of certain inventive concepts disclosed herein. In humans and other vertebrate animals, the heart generally comprises a muscular organ having four pumping chambers, wherein the flow thereof is at least partially controlled by various heart valves, namely, the aortic, mitral (or bicuspid), tricuspid, and pulmonary valves. The valves may be configured to open and close in response to a pressure gradient present during various stages of the cardiac cycle (e.g., relaxation and contraction) to at least partially control the flow of blood to a respective region of the heart and/or to blood vessels (e.g., pulmonary, aorta, etc.).

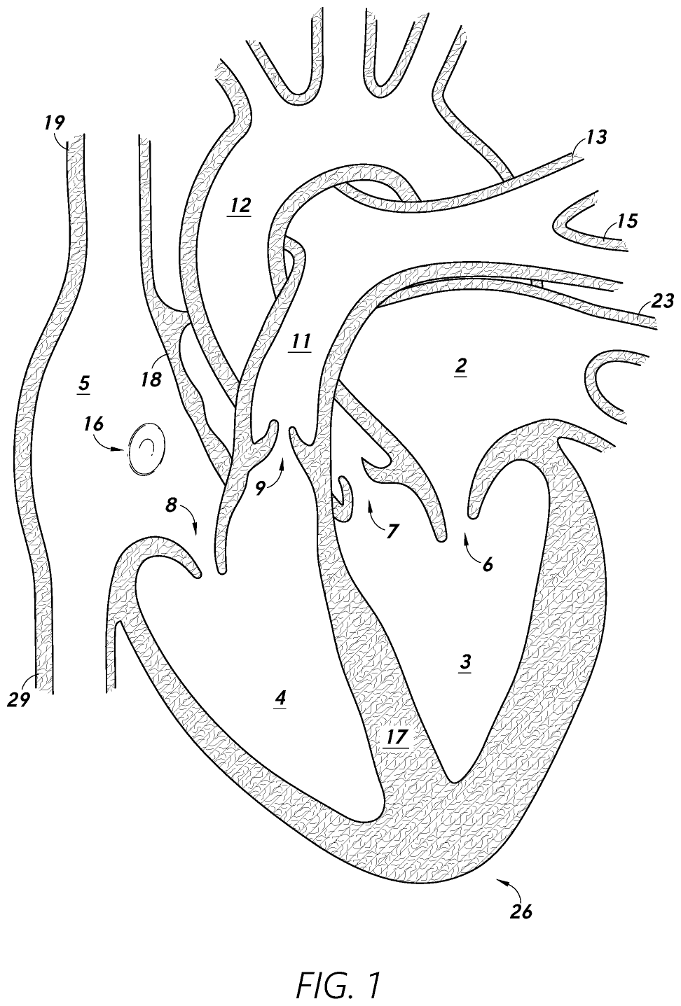

FIG. 1 illustrates an example representation of a heart 1 having various features relevant to certain examples of the present inventive disclosure. The heart 1 includes four chambers, namely the left atrium 2, the left ventricle 3, the right ventricle 4, and the right atrium 5. In terms of blood flow, blood generally flows from the right ventricle 4 into the pulmonary artery 11 via the pulmonary valve 9, which separates the right ventricle 4 from the pulmonary artery 11 and is configured to open during systole so that blood may be pumped toward the lungs and close during diastole to prevent blood from leaking back into the heart from the pulmonary artery 11. The pulmonary artery 11 carries deoxygenated blood from the right side of the heart to the lungs. The pulmonary artery 11 includes a pulmonary trunk and left 15 and right 13 pulmonary arteries that branch off of the pulmonary trunk, as shown. The pulmonary veins 23 carry blood from the lungs to the left atrium 2.

In addition to the pulmonary valve 9, the heart 1 includes three additional valves for aiding the circulation of blood therein, including the tricuspid valve 8, the aortic valve 7, and the mitral valve 6. The tricuspid valve 8 separates the right atrium 5 from the right ventricle 4. The tricuspid valve 8 generally has three cusps or leaflets and may generally close during ventricular contraction (i.e., systole) and open during ventricular expansion (i.e., diastole). The mitral valve 6 generally has two cusps/leaflets and separates the left atrium 2 from the left ventricle 3. The mitral valve 6 is configured to open during diastole so that blood in the left atrium 2 can flow into the left ventricle 3, and, when functioning properly, closes during systole to prevent blood from leaking back into the left atrium 2. The aortic valve 7 separates the left ventricle 3 from the aorta 12. The aortic valve 7 is configured to open during systole to allow blood leaving the left ventricle 3 to enter the aorta 12, and close during diastole to prevent blood from leaking back into the left ventricle 3.

The heart valves may generally comprise a relatively dense fibrous ring, referred to herein as the annulus, as well as a plurality of leaflets or cusps attached to the annulus. Generally, the size of the leaflets or cusps may be such that when the heart contracts the resulting increased blood pressure produced within the corresponding heart chamber forces the leaflets at least partially open to allow flow from the heart chamber. As the pressure in the heart chamber subsides, the pressure in the subsequent chamber or blood vessel may become dominant and press back against the leaflets. As a result, the leaflets/cusps come in apposition to each other, thereby closing the flow passage. Dysfunction of a heart valve and/or associated leaflets (e.g., pulmonary valve dysfunction) can result in valve leakage and/or other health complications.

The atrioventricular (i.e., mitral and tricuspid) heart valves may further comprise a collection of chordae tendineae and papillary muscles (not shown) for securing the leaflets of the respective valves to promote and/or facilitate proper coaptation of the valve leaflets and prevent prolapse thereof. The papillary muscles, for example, may generally comprise finger-like projections from the ventricle wall. The valve leaflets are connected to the papillary muscles by the chordae tendineae. A wall of muscle, referred to as the septum, separates the left-side chambers from the right-side chambers. In particular, an atrial septum wall portion 18 (referred to herein as the “atrial septum,” “interatrial septum,” or “septum”) separates the left atrium 2 from the right atrium 5, whereas a ventricular septum wall portion 17 (referred to herein as the “ventricular septum,” “interventricular septum,” or “septum”) separates the left ventricle 3 from the right ventricle 4. The inferior tip 26 of the heart 1 is referred to as the apex and is generally located on or near the midclavicular line, in the fifth intercostal space.

The coronary sinus 16 comprises a collection of veins joined together to form a large vessel that collects blood from the heart muscle (myocardium). The ostium of the coronary sinus, which can be guarded at least in part by a Thebesian valve in some patients, is open to the right atrium 5, as shown. The coronary sinus runs along a posterior aspect of the left atrium 2 and delivers less-oxygenated blood to the right atrium 5. The coronary sinus generally runs transversely in the left atrioventricular groove on the posterior side of the heart.

Any of several access pathways in the heart 1 may be utilized for maneuvering guidewires and catheters in and around the heart 1 to deploy implants and/or devices of the present application. For instance, access may be from above via either the subclavian vein or jugular vein into the superior vena cava (SVC) 19, right atrium 5, and from there into the coronary sinus 16. Alternatively, the access path may start in the femoral vein and through the inferior vena cava (IVC) 14 into the heart 1. Other access routes may also be used, and each can utilize a percutaneous incision through which the guidewire and catheter are inserted into the vasculature, normally through a sealed introducer, and from there the physician can control the distal ends of the devices from outside the body.

Health Conditions Associated with Cardiac Pressure and Other Parameters

As referenced above, certain physiological conditions or parameters associated with the cardiac anatomy can impact the health of a patient. For example, congestive heart failure is a condition associated with the relatively slow movement of blood through the heart and/or body, which causes the fluid pressure in one or more chambers of the heart to increase. As a result, the heart does not pump sufficient oxygen to meet the body's needs. The various chambers of the heart may respond to pressure increases by stretching to hold more blood to pump through the body or by becoming relatively stiff and/or thickened. The walls of the heart can eventually weaken and become unable to pump as efficiently. In some cases, the kidneys may respond to cardiac inefficiency by causing the body to retain fluid. Fluid build-up in arms, legs, ankles, feet, lungs, and/or other organs can cause the body to become congested, which is referred to as congestive heart failure. Acute decompensated congestive heart failure is a leading cause of morbidity and mortality, and therefore treatment and/or prevention of congestive heart failure is a significant concern in medical care.

The treatment and/or prevention of heart failure (e.g., congestive heart failure) can advantageously involve the monitoring of pressure in one or more chambers or regions of the heart or other anatomy. As described above, pressure buildup in one or more chambers or areas of the heart can be associated with congestive heart failure. Without direct or indirect monitoring of cardiac pressure, it can be difficult to infer, determine, or predict the presence or occurrence of congestive heart failure. For example, treatments or approaches not involving direct or indirect pressure monitoring may involve measuring or observing other present physiological conditions of the patient, such as measuring body weight, thoracic impedance, right heart catheterization, or the like. In some solutions, pulmonary capillary wedge pressure can be measured as a surrogate of left atrial pressure. For example, a pressure sensor may be disposed or implanted in the pulmonary artery, and readings associated therewith may be used as a surrogate for left atrial pressure. However, with respect to catheter-based pressure measurement in the pulmonary artery or certain other chambers or regions of the heart, use of invasive catheters may be required to maintain such pressure sensors, which may be uncomfortable or difficult to implement. Furthermore, certain lung-related conditions may affect pressure readings in the pulmonary artery, such that the correlation between pulmonary artery pressure and left atrial pressure may be undesirably attenuated. As an alternative to pulmonary artery pressure measurement, pressure measurements in the right ventricle outflow tract may relate to left atrial pressure as well. However, the correlation between such pressure readings and left atrial pressure may not be sufficiently strong to be utilized in congestive heart failure diagnostics, prevention, and/or treatment.

Additional solutions may be implemented for deriving or inferring left atrial pressure. For example, the E/A ratio, which is a marker of the function of the left ventricle of the heart representing the ratio of peak velocity blood flow from gravity in early diastole (the E wave) to peak velocity flow in late diastole caused by atrial contraction (the A wave), can be used as a surrogate for measuring left atrial pressure. The E/A ratio may be determined using echocardiography or other imaging technology; generally, abnormalities in the E/A ratio may suggest that the left ventricle cannot fill with blood properly in the period between contractions, which may lead to symptoms of heart failure, as explained above. However, E/A ratio determination generally does not provide absolute pressure measurement values.

Various methods for identifying and/or treating congestive heart failure involve the observation of worsening congestive heart failure symptoms and/or changes in body weight. However, such signs may appear relatively late and/or be relatively unreliable. For example, daily bodyweight measurements may vary significantly (e.g., up to 9% or more) and may be unreliable in signaling heart-related complications. Furthermore, treatments guided by monitoring signs, symptoms, weight, and/or other biomarkers have not been shown to substantially improve clinical outcomes. In addition, for patients that have been discharged, such treatments may necessitate remote telemedicine systems.

The present disclosure provides systems, devices, and methods for guiding the administration of medication relating to the treatment of congestive heart failure at least in part by directly monitoring pressure in the left atrium, or other chamber or vessel for which pressure measurements are indicative of left atrial pressure and/or pressure levels in one or more other vessels/chambers, such as for congestive heart failure patients in order to reduce hospital readmissions, morbidity, and/or otherwise improve the health prospects of the patient.

### Cardiac Pressure Monitoring

Cardiac pressure monitoring in accordance with examples of the present disclosure may provide a proactive intervention mechanism for preventing or treating congestive heart failure and/or other physiological conditions. Generally, increases in ventricular filling pressures associated with diastolic and/or systolic heart failure can occur prior to the occurrence of symptoms that lead to hospitalization. For example, cardiac pressure indicators may present weeks prior to hospitalization with respect to some patients. Therefore, pressure monitoring systems in accordance with examples of the present disclosure may advantageously be implemented to reduce instances of hospitalization by guiding the appropriate or desired titration and/or administration of medications before the onset of heart failure.

Dyspnea represents a cardiac pressure indicator characterized by shortness of breath or the feeling that one cannot breathe well enough. Dyspnea may result from elevated atrial pressure, which may cause fluid buildup in the lungs from pressure back-up. Pathological dyspnea can result from congestive heart failure. However, a significant amount of time may elapse between the time of initial pressure elevation and the onset of dyspnea, and therefore symptoms of dyspnea may not provide sufficiently-early signaling of elevated atrial pressure. By monitoring pressure directly according to examples of the present disclosure, normal ventricular filling pressures may advantageously be maintained, thereby preventing or reducing effects of heart failure, such as dyspnea.

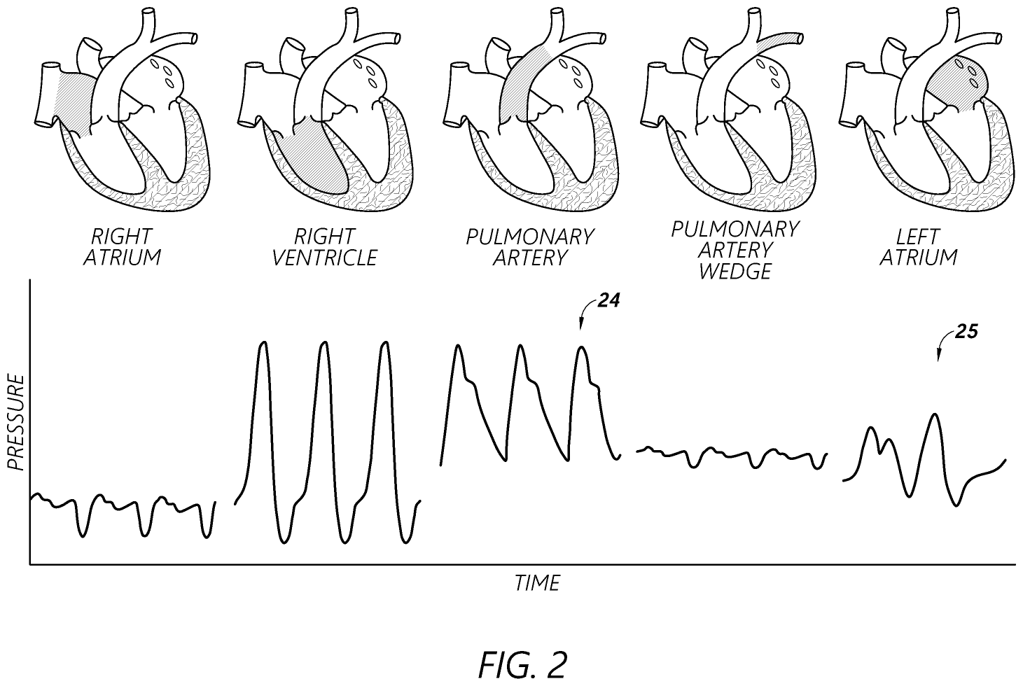

As referenced above, with respect to cardiac pressures, pressure elevation in the left atrium may be particularly correlated with heart failure. FIG. 2 illustrates example pressure waveforms associated with various chambers and vessels of the heart according to one or more examples. The various waveforms illustrated in FIG. 2 may represent waveforms obtained using right heart catheterization to advance one or more pressure sensors to the respective illustrated and labeled chambers or vessels of the heart. As illustrated in FIG. 2, the waveform 25, which represents left atrial pressure, may be considered to provide the best feedback for early detection of congestive heart failure. Furthermore, there may generally be a relatively strong correlation between increases and left atrial pressure and pulmonary congestion.

Left atrial pressure may generally correlate well with left ventricular end-diastolic pressure. However, although left atrial pressure and end-diastolic pulmonary artery pressure can have a significant correlation, such correlation may be weakened when the pulmonary vascular resistance becomes elevated. That is, pulmonary artery pressure generally fails to correlate adequately with left ventricular end-diastolic pressure in the presence of a variety of acute conditions, which may include certain patients with congestive heart failure. For example, pulmonary hypertension, which affects approximately 25% to 83% of patients with heart failure, can affect the reliability of pulmonary artery pressure measurement for estimating left-sided filling pressure. Therefore, pulmonary artery pressure measurement alone, as represented by the waveform 24, may be an insufficient or inaccurate indicator of left ventricular end-diastolic pressure, particularly for patients with co-morbidities, such as lung disease and/or thromboembolism. Left atrial pressure may further be correlated at least partially with the presence and/or degree of mitral regurgitation.

Left atrial pressure readings may be relatively less likely to be distorted or affected by other conditions, such as respiratory conditions or the like, compared to the other pressure waveforms shown in FIG. 2. Generally, left atrial pressure may be significantly predictive of heart failure, such as up two weeks before manifestation of heart failure. For example, increases in left atrial pressure, and both diastolic and systolic heart failure, may occur weeks prior to hospitalization, and therefore knowledge of such increases may be used to predict the onset of congestive heart failure, such as acute debilitating symptoms of congestive heart failure.

Cardiac pressure monitoring, such as left atrial pressure monitoring, can provide a mechanism to guide administration of medication to treat and/or prevent congestive heart failure. Such treatments may advantageously reduce hospital readmissions and morbidity, as well as provide other benefits. An implanted pressure sensor in accordance with examples of the present disclosure may be used to predict heart failure up two weeks or more before the manifestation of symptoms or markers of heart failure (e.g., dyspnea). When heart failure predictors are recognized using cardiac pressure sensor examples in accordance with the present disclosure, certain prophylactic measures may be implemented, including medication intervention, such as modification to a patient's medication regimen, which may help prevent or reduce the effects of cardiac dysfunction. Direct pressure measurement in the left atrium can advantageously provide an accurate indicator of pressure buildup that may lead to heart failure or other complications. For example, trends of atrial pressure elevation may be analyzed or used to determine or predict the onset of cardiac dysfunction, wherein drug or other therapy may be augmented to cause reduction in pressure and prevent or reduce further complications.

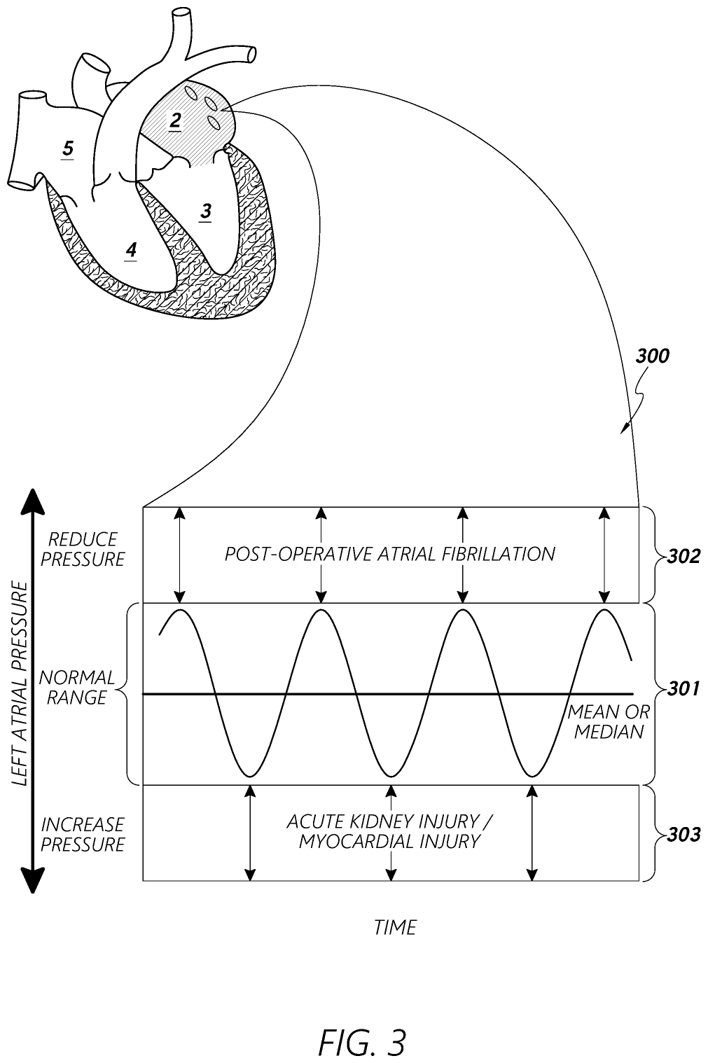

FIG. 3 illustrates a graph 300 showing left atrial pressure ranges including a normal range 301 of left atrial pressure that is not generally associated with substantial risk of postoperative atrial fibrillation, acute kidney injury, myocardial injury, heart failure and/or other health conditions. Examples of the present disclosure provide systems, devices, and methods for determining whether a patient's left atrial pressure is within the normal range 301, above the normal range 303, or below the normal range 302 through the use of certain sensor implant devices. For detected left atrial pressure above the normal range, which may be correlated with an increased risk of heart failure, examples of the present disclosure as described in detail below can inform efforts to reduce the left atrial pressure until it is brought within the normal range 301. Furthermore, for detected left atrial pressure that is below the normal range 301, which may be correlated with increased risks of acute kidney injury, myocardial injury, and/or other health complications, examples of the present disclosure as described in detail below can serve to facilitate efforts to increase the left atrial pressure to bring the pressure level within the normal range 301.

Implant Devices with Integrated Sensors

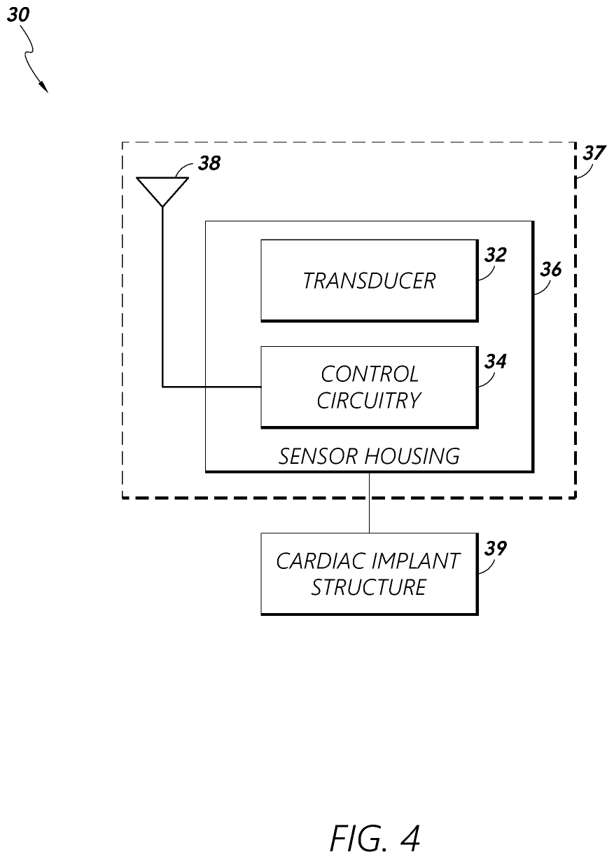

In some implementations, the present disclosure relates to sensors associated or integrated with cardiac shunts or other implant devices. Such integrated devices may be used to provide controlled and/or more effective therapies for treating and preventing heart failure and/or other health complications related to cardiac function. FIG. 4 is a block diagram illustrating an implant device 30 comprising a shunt (or other type of implant) structure 39. In some examples, the implant structure 39 is physically integrated with and/or connected to a sensor device 37. The sensor device 37 may be, for example, a pressure sensor, or other type of sensor. In some examples, the sensor 37 comprises a transducer 32, such as a pressure transducer, as well as certain control circuitry 34, which may be embodied in, for example, an application-specific integrated circuit (ASIC).

The control circuitry 34 may be configured to process signals received from the transducer 32 and/or communicate signals associated therewith wirelessly through biological tissue using the antenna 38. The term “control circuitry” is used herein according to its broad and ordinary meaning, and may refer to any collection of processors, processing circuitry, processing modules/units, chips, dies (e.g., semiconductor dies including come or more active and/or passive devices and/or connectivity circuitry), microprocessors, micro-controllers, digital signal processors, microcomputers, central processing units, field programmable gate arrays, programmable logic devices, state machines (e.g., hardware state machines), logic circuitry, analog circuitry, digital circuitry, and/or any device that manipulates signals (analog and/or digital) based on hard coding of the circuitry and/or operational instructions. Control circuitry referenced herein may further comprise one or more, storage devices, which may be embodied in a single memory device, a plurality of memory devices, and/or embedded circuitry of a device. Such data storage may comprise read-only memory, random access memory, volatile memory, non-volatile memory, static memory, dynamic memory, flash memory, cache memory, data storage registers, and/or any device that stores digital information. It should be noted that in examples in which control circuitry comprises a hardware and/or software state machine, analog circuitry, digital circuitry, and/or logic circuitry, data storage device(s)/register(s) storing any associated operational instructions may be embedded within, or external to, the circuitry comprising the state machine, analog circuitry, digital circuitry, and/or logic circuitry. The transducer(s) 32 and/or antenna(s) 38 can be considered part of the control circuitry 34.

The antenna 38 may comprise one or more coils or loops of conductive material, such as copper wire or the like. In some examples, at least a portion of the transducer 32, control circuitry 34, and/or the antenna 38 are at least partially disposed or contained within a sensor housing 36, which may comprise any type of material, and may advantageously be at least partially hermetically sealed. For example, the housing 36 may comprise glass or other rigid material in some examples, which may provide mechanical stability and/or protection for the components housed therein. In some examples, the housing 36 is at least partially flexible. For example, the housing may comprise polymer or other flexible structure/material, which may advantageously allow for folding, bending, or collapsing of the sensor 37 to allow for transportation thereof through a catheter or other introducing means.

The transducer 32 may comprise any type of sensor means or mechanism. For example, the transducer 32 may be a force-collector-type pressure sensor. In some examples, the transducer 32 comprises a diaphragm, piston, bourdon tube, bellows, or other strain- or deflection-measuring component(s) to measure strain or deflection applied over an area/surface thereof. The transducer 32 may be associated with the housing 36, such that at least a portion thereof is contained within or attached to the housing 36. With respect to sensor devices/components being “associated with” a stent or other implant structure, such terminology may refer to a sensor device or component being physically coupled, attached, or connected to, or integrated with, the implant structure.

In some examples, the transducer 32 comprises or is a component of a piezoresistive strain gauge, which may be configured to use a bonded or formed strain gauge to detect strain due to applied pressure, wherein resistance increases as pressure deforms the component/material. The transducer 32 may incorporate any type of material, including but not limited to silicon (e.g., monocrystalline), polysilicon thin film, bonded metal foil, thick film, silicon-on-sapphire, sputtered thin film, and/or the like.

In some examples, the transducer 32 comprises or is a component of a capacitive pressure sensor including a diaphragm and pressure cavity configured to form a variable capacitor to detect strain due to pressure applied to the diaphragm. The capacitance of the capacitive pressure sensor may generally decrease as pressure deforms the diaphragm. The diaphragm may comprise any material(s), including but not limited to metal, ceramic, silicon, and the like. In some examples, the transducer 32 comprises or is a component of an electromagnetic pressure sensor, which may be configured to measure the displacement of a diaphragm by means of changes in inductance, linear variable displacement transducer (LVDT) functionality, Hall Effect, or eddy current sensing. In some examples, the transducer 32 comprises or is a component of a piezoelectric strain sensor. For example, such a sensor may determine strain (e.g., pressure) on a sensing mechanism based on the piezoelectric effect in certain materials, such as quartz.

In some examples, the transducer 32 comprises or is a component of a strain gauge. For example, a strain gauge example may comprise a pressure sensitive element on or associated with an exposed surface of the transducer 32. In some examples, a metal strain gauge is adhered to a surface of the sensor, or a thin-film gauge may be applied on the sensor by sputtering or other technique. The measuring element or mechanism may comprise a diaphragm or metal foil. The transducer 32 may comprise any other type of sensor or pressure sensor, such as optical, potentiometric, resonant, thermal, ionization, or other types of strain or pressure sensors.

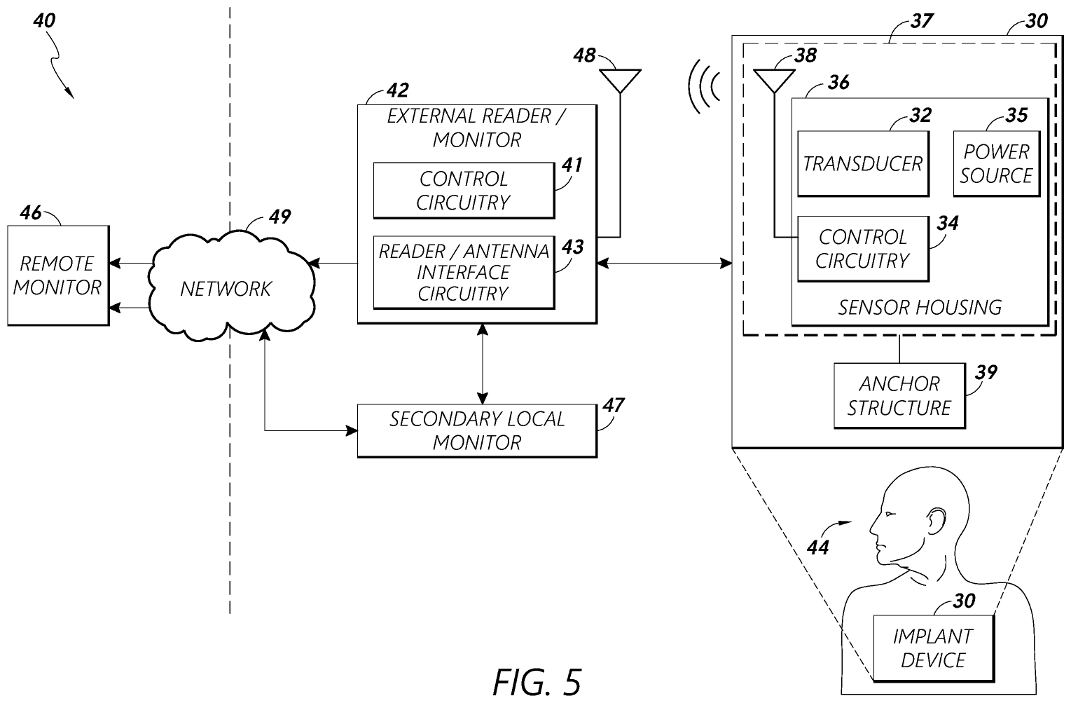

FIG. 5 shows a system 40 for monitoring one or more physiological parameters (e.g., left atrial pressure and/or volume) in a patient 44 according to one or more examples. The patient 44 can have a medical implant device 30 implanted in, for example, the heart (not shown), or associated physiology, of the patient 44. For example, the implant device 30 can be implanted at least partially within the left atrium and/or coronary sinus of the patient's heart. The implant device 30 can include one or more sensor transducers 32, such as one or more microelectromechanical system (MEMS) devices (e.g., MEMS pressure sensors, or other type of sensor transducer).

In certain examples, the monitoring system 40 can comprise at least two subsystems, including an implantable internal subsystem or device 30 that includes the sensor transducer(s) 32, as well as control circuitry 34 comprising one or more microcontroller(s), discrete electronic component(s), and one or more power and/or data transmitter(s) 38 (e.g., antennae coil). The monitoring system 40 can further include an external (e.g., non-implantable) subsystem that includes an external reader 42 (e.g., coil), which may include a wireless transceiver that is electrically and/or communicatively coupled to certain control circuitry 41. In certain examples, both the internal 30 and external 42 subsystems include a corresponding coil antenna for wireless communication and/or power delivery through patient tissue disposed therebetween. The sensor implant device 30 can be any type of implant device. For example, in some examples, the implant device 30 comprises a pressure sensor integrated with another functional implant structure 39, such as a prosthetic shunt or stent device/structure.

Certain details of the implant device 30 are illustrated in the enlarged block 30 shown. The implant device 30 can comprise an implant/anchor structure 39 as described herein. For example, the implant/anchor structure 39 can include a percutaneously-deliverable shunt device configured to be secured to and/or in a tissue wall to provide a flow path between two chambers and/or vessels of the heart, as described in detail throughout the present disclosure. In some examples, the anchor structure comprises one or more anchoring features configured to anchor the implant device 30 within a tissue wall. Although certain components are illustrated in FIG. 5 as part of the implant device 30, it should be understood that the sensor implant device 30 may only comprise a subset of the illustrated components/modules and can comprise additional components/modules not illustrated. The implant device may represent an example of the implant device shown in FIG. 4, and vice versa. The implant device 30 can advantageously include one or more sensor transducers 32, which can be configured to provide a response indicative of one or more physiological parameters of the patient 44, such as atrial pressure. Although pressure transducers are described, the sensor transducer(s) 32 can comprise any suitable or desirable types of sensor transducer(s) for providing signals relating to physiological parameters or conditions associated with the implant device 30 and/or patient 44.

The sensor transducer(s) 32 can comprise one or more MEMS sensors, optical sensors, piezoelectric sensors, electromagnetic sensors, strain sensors/gauges, accelerometers, gyroscopes, diaphragm-based sensors, and/or other types of sensors, which can be positioned in the patient 44 to sense one or more parameters relevant to the health of the patient. The transducer 32 may be a force-collector-type pressure sensor. In some examples, the transducer 32 comprises a diaphragm, piston, bourdon tube, bellows, or other strain- or deflection-measuring component(s) to measure strain or deflection applied over an area/surface thereof. The transducer 32 may be associated with the sensor housing 36, such that at least a portion thereof is contained within, or attached to, the housing 36.

In some examples, the transducer 32 comprises or is a component of a strain gauge, which may be configured to use a bonded or formed strain gauge to detect strain due to applied pressure. For example, the transducer 32 may comprise or be a component of a piezoresistive strain gauge, wherein resistance increases as pressure deforms the component/material of the strain gauge. The transducer 32 may incorporate any type of material, including but not limited to silicone, polymer, silicon (e.g., monocrystalline), polysilicon thin film, bonded metal foil, thick film, silicon-on-sapphire, sputtered thin film, and/or the like. In some examples, a metal strain gauge is adhered to the sensor surface, or a thin-film gauge may be applied on the sensor by sputtering or other technique. The measuring element or mechanism may comprise a diaphragm or metal foil. The transducer 32 may comprise any other type of sensor or pressure sensor, such as optical, potentiometric, resonant, thermal, ionization, or other types of strain or pressure sensors.

In some examples, the transducer 32 comprises or is a component of a capacitive pressure sensor including a diaphragm and pressure cavity configured to form a variable capacitor to detect strain due to pressure applied to the diaphragm. The capacitance of the capacitive pressure sensor may generally decrease as pressure deforms the diaphragm. The diaphragm may comprise any material(s), including but not limited to metal, ceramic, silicone, silicon or other semiconductor, and the like. In some examples, the transducer 32 comprises or is a component of an electromagnetic pressure sensor, which may be configured to measures the displacement of a diaphragm by means of changes in inductance, linear variable displacement transducer (LVDT) functionality, Hall Effect, or eddy current sensing. In some examples, the transducer 32 comprises or is a component of a piezoelectric strain sensor. For example, such a sensor may determine strain (e.g., pressure) on a sensing mechanism based on the piezoelectric effect in certain materials, such as quartz.

In some examples, the transducer(s) 32 is/are electrically and/or communicatively coupled to the control circuitry 34, which may comprise one or more application-specific integrated circuit (ASIC) microcontrollers or chips. The control circuitry 34 can further include one or more discrete electronic components, such as tuning capacitors, resistors, diodes, inductors, or the like.

In certain examples, the sensor transducer(s) 32 can be configured to generate electrical signals that can be wirelessly transmitted to a device outside the patient's body, such as the illustrated local external monitor system 42. In order to perform such wireless data transmission, the implant device 30 can include radio frequency (RF) (or other frequency band) transmission circuitry, such as signal processing circuitry and an antenna 38. The antenna 38 can comprise an antenna coil implanted within the patient. The control circuitry 34 may comprise any type of transceiver circuitry configured to transmit an electromagnetic signal, wherein the signal can be radiated by the antenna 38, which may comprise one or more conductive wires, coils, plates, or the like. The control circuitry 34 of the implant device 30 can comprise, for example, one or more chips or dies configured to perform some amount of processing on signals generated and/or transmitted using the device 30. However, due to size, cost, and/or other constraints, the implant device 30 may not include independent processing capability in some examples.

The wireless signals generated by the implant device 30 can be received by the local external monitor device or subsystem 42, which can include a reader/antenna-interface circuitry module 43 configured to receive the wireless signal transmissions from the implant device 30, which is disposed at least partially within the patient 44. For example, the module 43 may include transceiver device(s)/circuitry.

The external local monitor 42 can receive the wireless signal transmissions from the implant device 30 and/or provide wireless power to the implant device 30 using an external antenna 48, such as a wand device. The reader/antenna-interface circuitry 43 can include radio-frequency (RF) (or other frequency band) front-end circuitry configured to receive and amplify the signals from the implant device 30, wherein such circuitry can include one or more filters (e.g., band-pass filters), amplifiers (e.g., low-noise amplifiers), analog-to-digital converters (ADC) and/or digital control interface circuitry, phase-locked loop (PLL) circuitry, signal mixers, or the like. The reader/antenna-interface circuitry 43 can further be configured to transmit signals over a network 49 to a remote monitor subsystem or device 46. The RF circuitry of the reader/antenna-interface circuitry 43 can further include one or more of digital-to-analog converter (DAC) circuitry, power amplifiers, low-pass filters, antenna switch modules, antennas or the like for treatment/processing of transmitted signals over the network 49 and/or for receiving signals from the implant device 30. In certain examples, the local monitor 42 includes control circuitry 41 for performing processing of the signals received from the implant device 30. The local monitor 42 can be configured to communicate with the network 49 according to a known network protocol, such as Ethernet, Wi-Fi, or the like. In certain examples, the local monitor 42 comprises a smartphone, laptop computer, or other mobile computing device, or any other type of computing device.

In certain examples, the implant device 30 includes some amount of volatile and/or non-volatile data storage. For example, such data storage can comprise solid-state memory utilizing an array of floating-gate transistors, or the like. The control circuitry 34 may utilize data storage for storing sensed data collected over a period of time, wherein the stored data can be transmitted periodically to the local monitor 42 or other external subsystem. In certain examples, the implant device 30 does not include any data storage. The control circuitry 34 may be configured to facilitate wireless transmission of data generated by the sensor transducer(s) 32, or other data associated therewith. The control circuitry 34 may further be configured to receive input from one or more external subsystems, such as from the local monitor 42, or from a remote monitor 46 over, for example, the network 49. For example, the implant device 30 may be configured to receive signals that at least partially control the operation of the implant device 30, such as by activating/deactivating one or more components or sensors, or otherwise affecting operation or performance of the implant device 30.

The one or more components of the implant device 30 can be powered by one or more power sources 35. Due to size, cost and/or electrical complexity concerns, it may be desirable for the power source 35 to be relatively minimalistic in nature. For example, high-power driving voltages and/or currents in the implant device 30 may adversely affect or interfere with operation of the heart or other body part associated with the implant device. In certain examples, the power source 35 is at least partially passive in nature, such that power can be received from an external source wirelessly by passive circuitry of the implant device 30, such as through the use of short-range, or near-field wireless power transmission, or other electromagnetic coupling mechanism. For example, the local monitor 42 may serve as an initiator that actively generates an RF field that can provide power to the implant device 30, thereby allowing the power circuitry of the implant device to take a relatively simple form factor. In certain examples, the power source 35 can be configured to harvest energy from environmental sources, such as fluid flow, motion, or the like. Additionally or alternatively, the power source 35 can comprise a battery, which can advantageously be configured to provide enough power as needed over the monitoring period (e.g., 3, 5, 10, 20, 30, 40, or 90 days, or other period of time).

In some examples, the local monitor device 42 can serve as an intermediate communication device between the implant device 30 and the remote monitor 46. The local monitor device 42 can be a dedicated external unit designed to communicate with the implant device 30. For example, the local monitor device 42 can be a wearable communication device, or other device that can be readily disposed in proximity to the patient 44 and implant device 30. The local monitor device 42 can be configured to continuously, periodically, or sporadically interrogate the implant device 30 in order to extract or request sensor-based information therefrom. In certain examples, the local monitor 42 comprises a user interface, wherein a user can utilize the interface to view sensor data, request sensor data, or otherwise interact with the local monitor system 42 and/or implant device 30.

The system 40 can include a secondary local monitor 47, which can be, for example, a desktop computer or other computing device configured to provide a monitoring station or interface for viewing and/or interacting with the monitored cardiac pressure data. In an example, the local monitor 42 can be a wearable device or other device or system configured to be disposed in close physical proximity to the patient and/or implant device 30, wherein the local monitor 42 is primarily designed to receive/transmit signals to and/or from the implant device 30 and provide such signals to the secondary local monitor 47 for viewing, processing, and/or manipulation thereof. The external local monitor system 42 can be configured to receive and/or process certain metadata from or associated with the implant device 30, such as device ID or the like, which can also be provided over the data coupling from the implant device 30.

The remote monitor subsystem 46 can be any type of computing device or collection of computing devices configured to receive, process and/or present monitor data received over the network 49 from the local monitor device 42, secondary local monitor 47, and/or implant device 30. For example, the remote monitor subsystem 46 can advantageously be operated and/or controlled by a healthcare entity, such as a hospital, doctor, or other care entity associated with the patient 44. Although certain examples disclosed herein describe communication with the remote monitor subsystem 46 from the implant device indirectly through the local monitor device 42, in certain examples, the implant device 30 can comprise a transmitter capable of communicating over the network 49 with the remote monitor subsystem 46 without the necessity of relaying information through the local monitor device 42.

In some examples, at least a portion of the transducer 32, control circuitry 34, power source 35 and/or the antenna 38 are at least partially disposed or contained within the sensor housing 36, which may comprise any type of material, and may advantageously be at least partially hermetically sealed. For example, the housing 36 may comprise glass or other rigid material in some examples, which may provide mechanical stability and/or protection for the components housed therein. In some examples, the housing 36 is at least partially flexible. For example, the housing may comprise polymer or other flexible structure/material, which may advantageously allow for folding, bending, or collapsing of the sensor 37 to allow for transportation thereof through a catheter or other percutaneous introducing means.

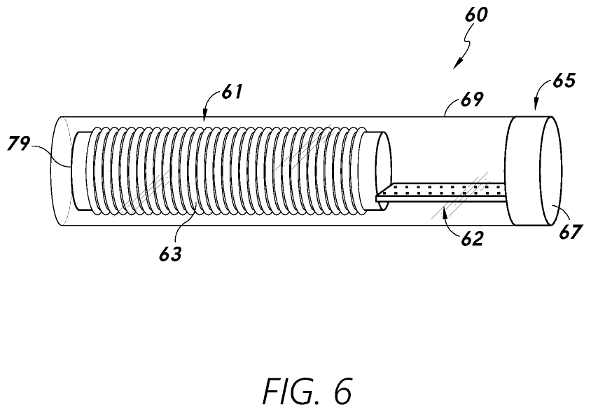

As referenced above, shunt and other implant devices/structures may be integrated with sensor, antenna/transceiver, and/or other components to facilitate in vivo monitoring of pressure and/or other physiological parameter(s). Sensor devices in accordance with examples of the present disclosure may be integrated with cardiac shunt structures/devices or other implant devices using any suitable or desirable anchoring or integration mechanism or configuration. FIG. 6 illustrates an example sensor assembly/device 60 that can be a component of a sensor implant device. The sensor device 60 may be configured to provide sensor readings relating to one or more physiological parameters associated with a target implantation site.

The sensor device 60 may be configured for anchoring to implant devices. For example, a coil form including one or more wires or other material or structure shaped into one or more winds of coil forming a fluid conduit/barrel portion and axial end flanges may be used to attach the sensor device 60 to one or more implants. A shunt structure may be integrated with pressure sensor functionality in accordance with certain examples disclosed herein. The shunt structure may be configured to hold the sensor device 60.

The sensor device 60 may advantageously be disposed, positioned, secured, oriented, and/or otherwise situated in a configuration in which a sensor transducer component 65 thereof is disposed within a channel area of a shunt structure. The term “channel area” is used herein according to its broad and ordinary meaning and may refer to a three-dimensional space defined by a radial boundary of a fluid conduit and extending axially from the fluid conduit.

In some examples, the sensor assembly 61 includes a sensor component 65 and an antenna component 69. The sensor component 65 may comprise any type of sensor device as described in detail above. In some examples, the sensor 65 may be attached to or integrated with an arm member of a shunt structure.

The sensor 65 includes a sensor element 67, such as a pressure sensor transducer. As described herein, the sensor assembly 61 may be configured to implement wireless data and/or power transmission. The sensor assembly 61 may include an antenna component 69 for such purpose. The antenna 69 may be contained at least partially within an antenna housing 79, which may further have disposed therein certain control circuitry configured to facilitate wireless data and/or power communication functionality. In some examples, the antenna component 69 comprises one or more conductive coils 62, which may facilitate inductive powering and/or data transmission. In examples comprising conductive coil(s), such coil(s) may be wrapped/disposed at least partially around a magnetic (e.g., ferrite, iron) core 63.

The antenna component 69 may be attached to, integrated with, or otherwise associated with an arm/anchor feature of a shunt structure

The sensor assembly 61 may advantageously be biocompatible. For example, the sensor 65 and antenna 69 may comprise biocompatible housings, such as a housing comprising glass or other biocompatible material. However, at least a portion of the sensor element 67, such as a diaphragm or other component, may be exposed to the external environment in some examples in order to allow for pressure readings, or other parameter sensing, to be implemented. With respect to the antenna housing 79, the housing 79 may comprise an at least partially rigid cylindrical or tube-like form, such as a glass cylinder form. In some examples, the sensor 65/67 component is approximately 3 mm or less in diameter. The antenna 69 may be approximately 20 mm or less in length.

The sensor assembly 61 may be configured to communicate with an external system when implanted in a heart or other area of a patient's body. For example, the antenna 69 may receive power wirelessly from the external system and/or communicate sensed data or waveforms to and/or from the external system. The sensor assembly 61 may be attached to, or integrated with, a shunt structure in any suitable or desirable way. For example, in some implementations, the sensor 65 and/or antenna 69 may be attached or integrated with the shunt structure using mechanical anchoring means. In some examples, the sensor 65 and/or antenna 69 may be contained in a pouch or other receptacle that is attached to a shunt structure.

The sensor element 67 may comprise a pressure transducer. For example, the pressure transducer may be a microelectromechanical system (MEMS) transducer comprising a semiconductor diaphragm component. In some examples, the transducer may include an at least partially flexible or compressible diaphragm component, which may be made from silicone or other flexible material. The diaphragm component may be configured to be flexed or compressed in response to changes in environmental pressure.

### Sensor Implant Devices

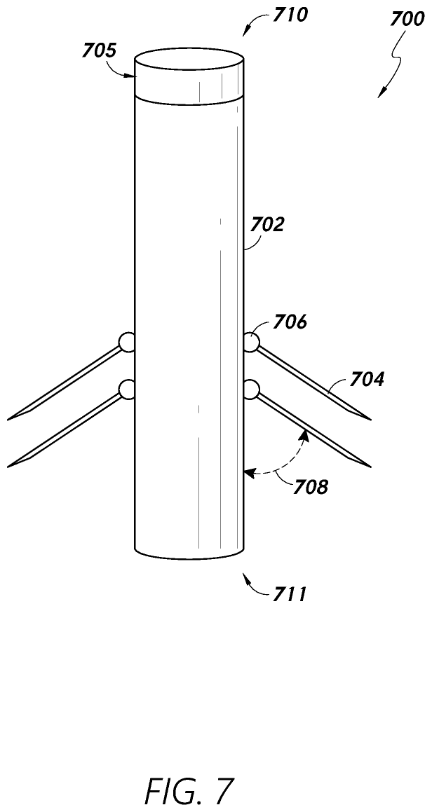

FIG. 7 illustrates a sensor implant device 700 comprising a sensor body/device 702 and/or one or more anchoring features 704, in accordance with one or more examples. The sensor body 702 can be coupled, attached, and/or otherwise releasably and/or permanently secured to the one or more anchoring features 704. The one or more anchoring features can comprise one or more needles, clips, puncture coils, hooks, arms, cords, pins, grooves, protrusions, pegs, spikes, and/or other features configured for anchoring and/or anchoring at one or more areas of tissue within a heart. In some examples, the one or more anchoring features 704 may be configured to anchor the sensor implant device 700 at least partially within a tissue wall.

The sensor device 702 may comprise at least one sensor component 705. The sensor component(s) 705 may comprise any type of sensor element as described in detail above. The sensor implant device 700 may be configured to position the sensor component(s) 705 at a target location within a body, which can include a heart chamber (e.g., a left atrium), an opening (e.g., a left atrial appendage) into and/or from a heart chamber, and/or a blood flow pathway (e.g., a coronary sinus).

In some examples, the one or more anchoring features 704 may be configured to extend in multiple directions around the sensor device 702. The one or more anchoring features 704 may be configured to extend at an approximately 90° angle with respect to each other from the sensor device 702. Additionally or alternatively, one or more anchoring features 704 may extend in generally opposite directions laterally (i.e., along a diameter of the sensor device 702) from the sensor device 702. The one or more anchoring features 704 may extend linearly and/or non-linearly away from and/or along the sensor device 702.

The one or more anchoring features 704 may be configured to form a hinged attachment to and/or to extend from one or more joints 706 (e.g., hinges and/or hinge joints) attached to and/or extending from the sensor device 702. In some examples, each joint 706 may be associated with a corresponding anchoring feature 704 of the sensor implant device 700. In some examples, a joint 706 may be configured to enable adjustment of an angle 708 between an anchoring feature 704 and the sensor device 702. For example, the one or more anchoring features 704 may be configured to freely and/or manually swing and/or hinge between a collapsed configuration/form (e.g., approximately flat/flush and/or in parallel with a length and/or surface of the sensor device 702) and/or an expanded configuration/form (e.g., approximately at a 45° angle with a surface of the sensor device 702). In some examples, the one or more anchoring features 704 may not expand beyond a given angle 708 (e.g., may not expand beyond a 45° or 90° angle with respect to a surface of the sensor device 702). One or more anchoring features 704 may be angled in the expanded configuration to allow the one or more anchoring features 704 to effectively embed into tissue surrounding the sensor implant device 700 and/or to prevent the sensor implant device 700 from being dislodged from tissue.

In some examples, the one or more anchoring features 704 may be biased in the collapsed form and/or in the expanded form. For example, the one or more anchoring features 704 may be biased in the expanded form shown in FIG. 7. The one or more anchoring features 704 may be configured to bend and/or swing to a compressed form while within a catheter and/or other delivery device and/or may be configured to naturally assume the expanded form upon and/or following removal from the catheter and/or other delivery device. In some examples, the one or more anchoring features 704 may be configured to expand to the expanded form while within a tissue wall. Expansion of the one or more anchoring features 704 may be activated by the sensor implant device 700 moving in an opposite direction of the direction of insertion into the tissue wall.

While the sensor implant device 700 is shown in FIG. 7 comprising four anchoring features 704, the sensor implant device 700 may comprise any number of anchoring features 704. In some examples, the sensor implant device 700 may comprise a first set of two anchoring features 704 at a first side of the sensor device 702 and/or a second set of two anchoring features 704 at a second side of the sensor device 702.

In some examples, the sensor body 702 may comprise one or more pointed ends to facilitate puncturing and/or driving the sensor body 702 into a tissue wall. Additionally or alternatively, the sensor body 702 may be delivered via a catheter and/or similar device having a pointed tip and/or configured to pierce, embed into, and/or drive through a tissue wall.

While the sensor body 702 comprises a single sensor component 705 in FIG. 7, the sensor body 702 can comprise any number of sensor components 705. For example, the sensor body 702 can comprise a first sensor component 705 at a first end 710 of the sensor body 702 and/or a second sensor component 705 at a second end 711 of the sensor body 702. In some examples, the sensor body 702 may be separate from the sensor component 705 and/or the sensor body 702 and sensor component 705 may be interconnected via wiring.

In some examples, the sensor implant device 700 may be configured for anchoring within a tissue wall between a first heart chamber/blood flow pathway and a second heart chamber/blood flow pathway. For example, the sensor implant device 700 may be configured for anchoring within a tissue wall separating a left atrium and a coronary sinus. The sensor implant device 700 can comprise a first sensor component 705 to obtain measurements at the left atrium and/or a second sensor component 705 to obtain measurements at the coronary sinus.

In some examples, the sensor device 702 and/or sensor component 705 may form a generally cylindrical and/or tubular form having generally linear/straight sides. However, the sensor device 702 may have an uneven surface and/or may comprise one or more pegs and/or portions having an increased diameter with respect to other portions of the sensor device 702. For example, the sensor component 705 may have an increased diameter relative to other portions of the sensor device 702. In this way, the sensor component 705 may advantageously be prevented from becoming embedded in a tissue wall.

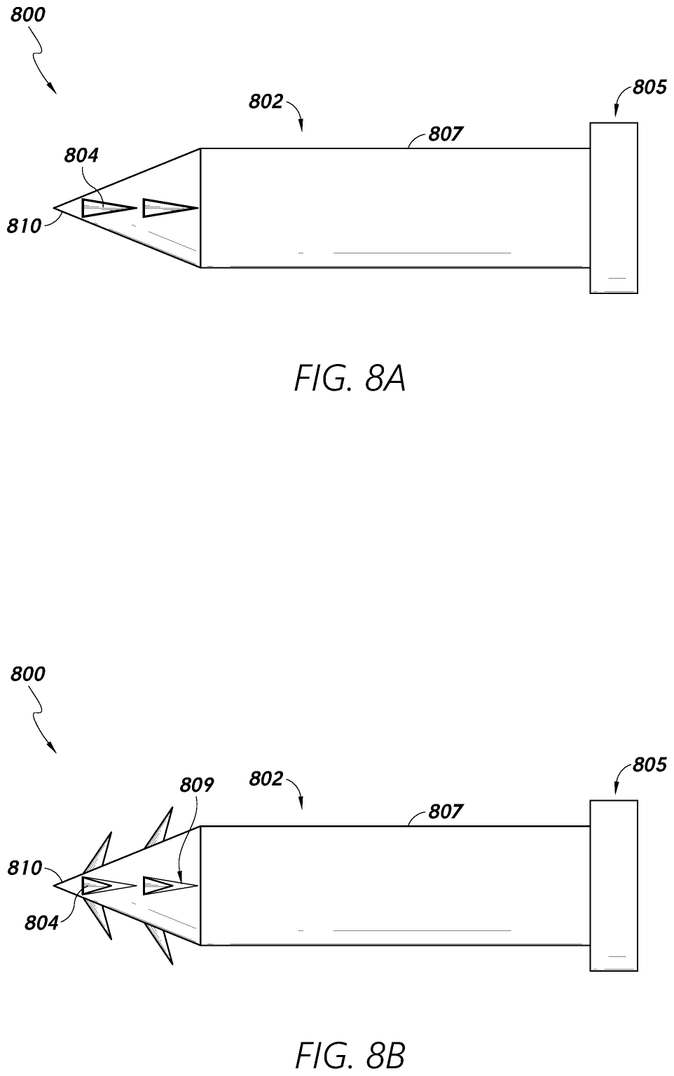

FIGS. 8A and 8B illustrate a sensor implant device 800 comprising a sensor body/device 802 and/or one or more anchoring features 804, in accordance with one or more examples. In some examples, the one or more anchoring features 804 may be configured to fit into and/or extend from corresponding receptors 809, cavities, and/or indentations in the sensor device 802. The one or more anchoring features can comprise one or more needles, clips, puncture coils, hooks, arms, cords, spikes, protrusions, pegs, and/or other features configured for anchoring and/or anchoring at one or more areas of tissue within a heart.

FIG. 8A illustrates a collapsed/compressed form of the sensor implant device 800. In the compressed form, the one or more anchoring features 804 may be situated at least partially within the receptors 809 of the sensor body 802 (e.g., at an end portion 810 of the sensor body 802) to reduce the profile of the sensor implant device 800. The sensor implant device 800 may be configured to assume the compressed form while within a catheter and/or other delivery device(s). In some examples, the one or more anchoring features 804 may be coupled to the sensor body 802 via a hinged attachment and/or hinges and/or similar mechanisms to allow the one or more anchoring features 804 to form moveable attachments to the sensor body 802.

As shown in FIG. 8A, in the unexpanded form, the one or more anchoring features 804 may extend generally towards the sensor component 805. For example, the anchoring features 804 may extend generally along a surface of the sensor body 802 and/or along a surface of the end portion 810 towards a second end of the sensor body 802 and/or towards the sensor component 805.

FIG. 8B illustrates an expanded form of the sensor implant device 800. In the expanded form, the one or more anchoring features 804 may extend away from the sensor device 802. For example, the one or more anchoring features 804 may assume a generally perpendicular and/or 45° angle with respect to at least a portion of the sensor device 802. The one or more anchoring features 804 may be configured to expand to no more than a 90° angle with respect to a surface of the sensor body 802. In some examples, at least partial removal of the sensor implant device 800 from a catheter and/or other delivery system(s) may cause activation and/or extension of the one or more anchoring features 804.

In some examples, the one or more anchoring features 804 (including anchoring features of any of the various sensor implant devices described herein) may be configured to swing freely between an expanded form and a compressed/unexpanded form. Additionally or alternatively, the one or more anchoring features 804 may be spring-loaded and/or otherwise biased to the expanded form. For example, one or more springs may be situated within the receptors 809 to press the anchoring features 804 out of the receptors 809.

The sensor device 802 may comprise at least one sensor component 805. The sensor component(s) 805 may comprise any type of sensor device as described in detail above. The sensor implant device 800 may be configured to position the sensor component(s) 805 at a target location within a body, which can include a heart chamber (e.g., a left atrium), an opening (e.g., a left atrial appendage) into and/or from a heart chamber, and/or a blood flow pathway (e.g., a coronary sinus).

In some examples, the one or more anchoring features 804 may be configured to extend in multiple directions around the sensor body 802. The one or more anchoring features 804 may extend in generally opposite directions laterally (i.e., along a diameter of the sensor body 802) from the sensor body 802. The one or more anchoring features 804 may extend linearly and/or non-linearly away from and/or along the sensor body 802.

The one or more anchoring features 804 may be configured to swing and/or hinge between a collapsed configuration (e.g., approximately in parallel with a conical surface of an end portion 810 of the sensor body 802) and/or an expanded configuration (e.g., approximately at a 45° angle with the conical surface of the end portion 810 of the sensor body 802). One or more anchoring features 804 may be angled in the expanded configuration to allow the one or more anchoring features 804 to effectively embed into tissue surrounding the sensor implant device 800 and/or to prevent the sensor implant device 800 from being dislodged from tissue.

The sensor implant device 800 may comprise any number of anchoring features 804. In some examples, the sensor implant device 800 may comprise a first set of two anchoring features 804 at a first side of the sensor device 802, a second set of two anchoring features 804 at a second side of the sensor device 802, and/or a third set of two anchoring features 804 at a third side of the sensor device 802.

In some examples, the sensor body 802 may have one or more pointed ends to facilitate driving the sensor device 802 into a tissue wall. For example, the sensor body 802 may comprise a pointed and/or conical end portion 810 having a pointed tip. The conical end portion 810 may be situated at an opposite side/end of the sensor body 802 than the sensor component 805, which can be situated at a second end of the sensor device 802. Additionally or alternatively, the sensor body 802 may be delivered via a catheter and/or similar device having a pointed tip and/or may be configured to pierce, embed into, and/or drive through a tissue wall. In some examples, the sensor device 802 may further comprise a body portion 807 situated between the conical end portion 810 and a sensor component 805. At least a portion of the one or more anchoring features 804 may be coupled to and/or within the conical end portion 810 of the sensor device 802.

While the sensor device 802 comprises a single sensor component 805 in FIGS. 8A and 8B, the sensor device 802 can comprise any number of sensor components 805. For example, the sensor device 802 can comprise a first sensor component 805 at a first end portion 810 of the sensor device 802 and/or a second sensor component 805 at a second end portion of the sensor device 802. In some examples, the sensor implant device 800 may be configured for anchoring within a tissue wall between a first heart chamber/blood flow pathway and a second heart chamber/blood flow pathway. For example, the sensor implant device 800 may be configured for anchoring within a tissue wall separating a left atrium and a coronary sinus. The sensor implant device 800 can comprise a first sensor component 805 to obtain measurements at the left atrium and/or a second sensor component 805 to obtain measurements at the coronary sinus.

In some examples, the sensor device 802 and/or sensor component 805 may form a generally cylindrical and/or tubular form having generally linear/straight sides. However, the sensor device 802 may have an uneven surface and/or may comprise one or more pegs and/or portions having a greater diameter/width with than portions of the sensor device 802. For example, the sensor component 805 may have a greater diameter/width than the body portion 807 and/or conical end portion 810 of the sensor device 802. In this way, the sensor component 805 may advantageously be prevented from becoming embedded in a tissue wall.

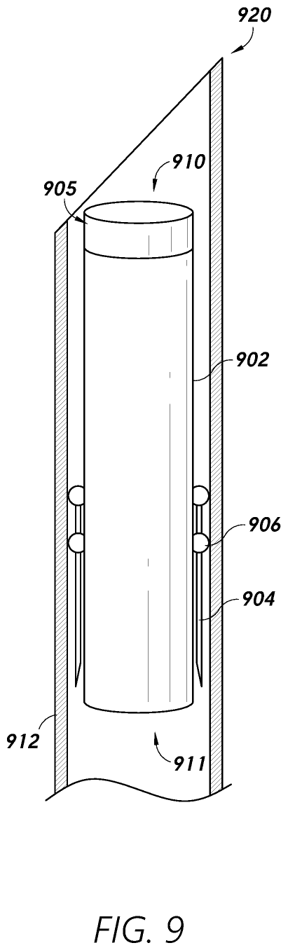

FIG. 9 illustrates a sensor implant device delivered via a catheter 912, in accordance with one or more examples. The sensor implant device can comprise a sensor body/device 902 and/or one or more anchoring features 904. The sensor body 902 can be coupled, attached, and/or otherwise releasably and/or permanently secured to the one or more anchoring features 904. The one or more anchoring features can comprise one or more needles, clips, puncture coils, hooks, arms, cords, protrusions, spikes, pegs, and/or other features configured for anchoring and/or anchoring at one or more areas of tissue within a heart.

In some examples, the one or more anchoring features 904 may be configured to assume a compressed and/or unexpanded form while within the catheter 912, as shown in FIG. 9. In the compressed/unexpanded form, the one or more anchoring features 904 may be configured to lay and/or be positioned flatly/flushly against an outer surface of the sensor device 902 and/or may be configured to enter one or more receptors of the sensor device 902.

The sensor device 902 may comprise at least one sensor component 905. The sensor component(s) 905 may comprise any type of sensor element as described in detail above. The sensor implant device may be configured to position the sensor component(s) 905 at a target location within a body, which can include a heart chamber (e.g., a left atrium), an opening (e.g., a left atrial appendage) into and/or from a heart chamber, and/or a blood flow pathway (e.g., a coronary sinus).

In some examples, the one or more anchoring features 904 may be configured to extend in multiple directions around the sensor device 902. The one or more anchoring features 904 may be configured to extend at an approximately 90° angle with respect to each other from the sensor device 902. Additionally or alternatively, one or more anchoring features 904 may extend in generally opposite directions laterally (i.e., along a diameter of the sensor device 902) from the sensor device 902. The one or more anchoring features 904 may extend linearly and/or non-linearly away from and/or along the sensor device 902.

The one or more anchoring features 904 may be configured to couple to and/or extend from one or more joints 906 attached to and/or extending from the sensor device 902. In some examples, each joint 906 may be associated with a corresponding anchoring feature 904 of the sensor implant device. In some examples, a joint 906 may be configured to enable adjustment of an angle between an anchoring feature 904 and the sensor device 902. For example, the one or more anchoring features 904 may be configured to swing and/or hinge between a collapsed configuration (e.g., approximately in parallel with a length of the sensor device 902) and/or an expanded configuration (e.g., approximately at a 45° angle with the sensor device 902). One or more anchoring features 904 may be angled in the expanded configuration to allow the one or more anchoring features 904 to effectively embed into tissue surrounding the sensor implant device and/or to prevent the sensor implant device from being dislodged from tissue.

While the sensor implant device is shown in FIG. 9 comprising four anchoring features 904, the sensor implant device may comprise any number of anchoring features 904. In some examples, the sensor implant device may comprise a first set of two anchoring features 904 at a first side of the sensor device 902 and/or a second set of two anchoring features 904 at a second side of the sensor device 902.

In some examples, the sensor device 902 may one or more pointed ends to facilitate driving the sensor device 902 into a tissue wall. Additionally or alternatively, the sensor device 902 may be delivered via a catheter 912 and/or similar device having a pointed tip 920 and/or configured to pierce, embed into, and/or drive through a tissue wall.

While the sensor device 902 comprises a single sensor component 905 in FIG. 9, the sensor device 902 can comprise any number of sensor components 905. For example, the sensor device 902 can comprise a first sensor component 905 at a first end 910 of the sensor device 902 and/or a second sensor component 905 at a second end 911 of the sensor device 902. In some examples, the sensor implant device may be configured for anchoring within a tissue wall between a first heart chamber/blood flow pathway and a second heart chamber/blood flow pathway. For example, the sensor implant device may be configured for anchoring within a tissue wall separating a left atrium and a coronary sinus. The sensor implant device can comprise a first sensor component 905 to obtain measurements at the left atrium and/or a second sensor component 905 to obtain measurements at the coronary sinus.

In some examples, the sensor device 902 and/or sensor component 905 may form a generally cylindrical and/or tubular form having generally linear/straight sides. However, the sensor device 902 may have an uneven surface and/or may comprise one or more pegs and/or portions having an increased diameter with respect to other portions of the sensor device 902. For example, the sensor component 905 may have an increased diameter compared to other portions of the sensor device 902. In this way, the sensor component 905 may advantageously be prevented from becoming embedded in a tissue wall.

As shown in FIG. 9, in the unexpanded form, the one or more anchoring features 904 may extend generally away from the sensor component 905. For example, the one or more anchoring features 904 may be configured to extend along a surface of the sensor body 902 towards a second end 911 of the sensor body 902 and/or the sensor component 910 may be situated at or near a first end 910 of the sensor body 902.

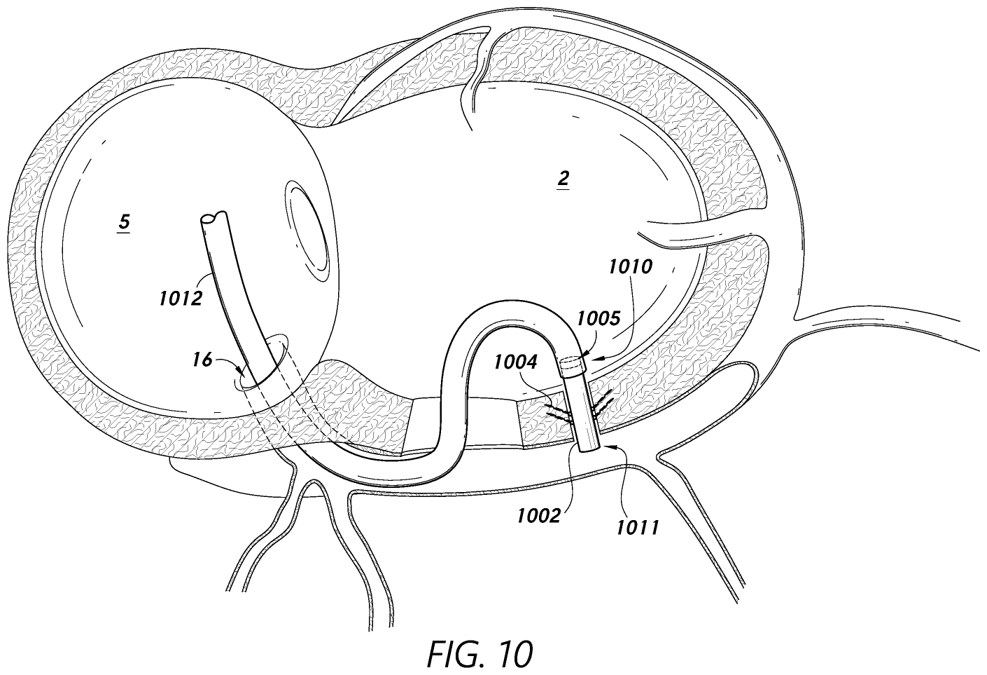

FIG. 10 illustrates a delivery process for delivering a sensor implant device via a catheter 1012 to a tissue wall of a left atrium 2, in accordance with one or more examples. The sensor implant device can comprise a sensor body/device 1002 and/or one or more anchoring features 1004. The sensor device 1002 can be coupled, attached, and/or otherwise releasably and/or permanently secured to the one or more anchoring features 1004. The one or more anchoring features can comprise one or more needles, clips, puncture coils, hooks, arms, cords, and/or other features configured for anchoring and/or anchoring at one or more areas of tissue within a heart.

The sensor device 1002 may comprise at least one sensor component. The sensor component(s) may comprise any type of sensor element as described in detail above. The sensor implant device may be configured to position the sensor component(s) at a target location within a body, which can include a heart chamber (e.g., a left atrium), an opening (e.g., a left atrial appendage) into and/or from a heart chamber, and/or a blood flow pathway (e.g., a coronary sinus).

In some examples, the one or more anchoring features 1004 may be configured to extend in multiple directions around the sensor device 1002. The one or more anchoring features 1004 may be configured to extend at an approximately 90° angle with respect to each other along an exterior surface of the sensor device 1002. Additionally or alternatively, one or more anchoring features 1004 may extend in generally opposite directions laterally (i.e., along a diameter of the sensor device 1002) from the sensor device 1002. The one or more anchoring features 1004 may extend linearly and/or non-linearly away from and/or along the sensor device 1002.

The one or more anchoring features 1004 may be configured to couple to and/or extend from one or more joints attached to and/or extending from the sensor device 1002. In some examples, each joint may be associated with a corresponding anchoring feature 1004 of the sensor implant device. In some examples, a joint may be configured to enable adjustment of an angle between an anchoring feature 1004 and the sensor device 1002. For example, the one or more anchoring features 1004 may be configured to swing and/or hinge between a collapsed configuration (e.g., approximately in parallel with a length of the sensor device 1002) and/or an expanded configuration (e.g., approximately at a 45° angle with the sensor device 1002). One or more anchoring features 1004 may be angled in the expanded configuration to allow the one or more anchoring features 1004 to effectively embed into tissue surrounding the sensor implant device and/or to prevent the sensor implant device from being dislodged from tissue.

While the sensor implant device is shown in FIG. 10 comprising four anchoring features 1004, the sensor implant device may comprise any number of anchoring features 1004. In some examples, the sensor implant device may comprise a first set of two anchoring features 1004 at a first side of the sensor device 1002 and/or a second set of two anchoring features 1004 at a second side of the sensor device 1002.

In some examples, the sensor device 1002 may comprise one or more pointed ends to facilitate driving the sensor device 1002 into a tissue wall. Additionally or alternatively, the sensor device 1002 may be delivered via a catheter 1012 and/or similar device having a pointed tip and/or configured to pierce, embed into, and/or drive through a tissue wall.

While the sensor device 1002 comprises a single sensor component 1005 in FIG. 10, the sensor device 1002 can comprise any number of sensor components 1005. For example, the sensor device 1002 can comprise a first sensor component 1005 at a first end 1010 of the sensor device 1002 and/or a second sensor component 1005 at a second end 1011 of the sensor device 1002. In some examples, the sensor implant device may be configured for anchoring within a tissue wall between a first heart chamber/blood flow pathway and a second heart chamber/blood flow pathway. For example, the sensor implant device may be configured for anchoring within a tissue wall separating a left atrium and a coronary sinus. The sensor implant device can comprise a first sensor component 1005 to obtain measurements at the left atrium and/or a second sensor component 1005 to obtain measurements at the coronary sinus.

In some examples, the sensor device 1002 and/or sensor component 1005 may form a generally cylindrical and/or tubular form having generally linear/straight sides. However, the sensor device 1002 may have an uneven surface and/or may comprise one or more pegs and/or portions having an increased diameter with respect to other portions of the sensor device 1002. For example, the sensor component 1005 may have an increased diameter compared to other portions of the sensor device 1002. In this way, the sensor component 1005 may advantageously be prevented from becoming embedded in a tissue wall.

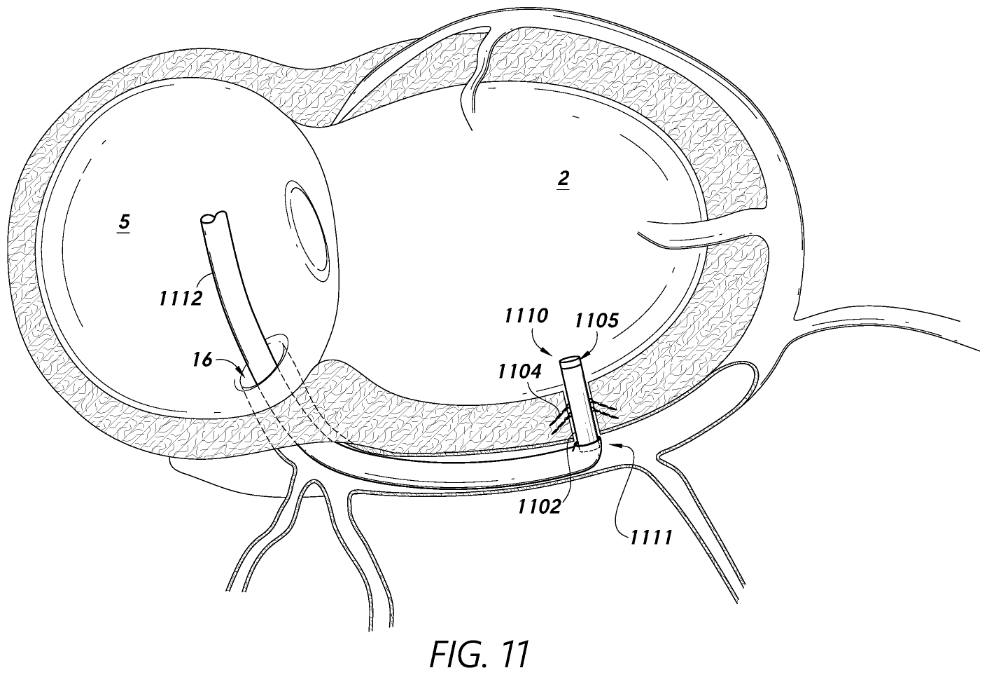

FIG. 11 illustrates a delivery process for delivering a sensor implant device via a catheter 1012 to a tissue wall of a coronary sinus 16, in accordance with one or more examples. The sensor implant device can comprise a sensor body/device 1102 and/or one or more anchoring features 1104. The sensor device 1102 can be coupled, attached, and/or otherwise releasably and/or permanently secured to the one or more anchoring features 1104. The one or more anchoring features can comprise one or more needles, clips, puncture coils, hooks, arms, cords, protrusions, spikes, pegs, and/or other features configured for anchoring and/or anchoring at one or more areas of tissue within a heart.

The sensor device 1102 may comprise at least one sensor component. The sensor component(s) may comprise any type of sensor element as described in detail above. The sensor implant device may be configured to position the sensor component(s) at a target location within a body, which can include a heart chamber (e.g., a left atrium), an opening (e.g., a left atrial appendage) into and/or from a heart chamber, and/or a blood flow pathway (e.g., a coronary sinus).

In some examples, the one or more anchoring features 1104 may be configured to extend in multiple directions around the sensor device 1102. The one or more anchoring features 1104 may be configured to extend at an approximately 90° angle with respect to each other from the sensor device 1102. Additionally or alternatively, one or more anchoring features 1104 may extend in generally opposite directions laterally (i.e., along a diameter of the sensor device 1102) from the sensor device 1102. The one or more anchoring features 1104 may extend linearly and/or non-linearly away from and/or along the sensor device 1102.

The one or more anchoring features 1104 may be configured to couple to and/or extend from one or more joints attached to and/or extending from the sensor device 1102. In some examples, each joint may be associated with a corresponding anchoring feature 1104 of the sensor implant device. In some examples, a joint may be configured to enable adjustment of an angle between an anchoring feature 1104 and the sensor device 1102. For example, the one or more anchoring features 1104 may be configured to swing and/or hinge between a collapsed configuration (e.g., approximately in parallel with a length of the sensor device 1102) and/or an expanded configuration (e.g., approximately at a 45° angle with the sensor device 1102). One or more anchoring features 1104 may be angled in the expanded configuration to allow the one or more anchoring features 1104 to effectively embed into tissue surrounding the sensor implant device and/or to prevent the sensor implant device from being dislodged from tissue.

While the sensor implant device is shown in FIG. 11 comprising four anchoring features 1104, the sensor implant device may comprise any number of anchoring features 1104. In some examples, the sensor implant device may comprise a first set of two anchoring features 1104 at a first side of the sensor device 1102 and/or a second set of two anchoring features 1104 at a second side of the sensor device 1102.

In some examples, the sensor device 1102 may comprise one or more pointed ends to facilitate driving the sensor device 1102 into a tissue wall. Additionally or alternatively, the sensor device 1102 may be delivered via a catheter 1112 and/or similar device having a pointed tip and/or configured to pierce, embed into, and/or drive through a tissue wall.

While the sensor device 1102 comprises a single sensor component 1105 in FIG. 11, the sensor device 1102 can comprise any number of sensor components 1105. For example, the sensor device 1102 can comprise a first sensor component 1105 at a first end 1110 of the sensor device 1102 and/or a second sensor component 1105 at a second end 1111 of the sensor device 1102. In some examples, the sensor implant device may be configured for anchoring within a tissue wall between a first heart chamber/blood flow pathway and a second heart chamber/blood flow pathway. For example, the sensor implant device may be configured for anchoring within a tissue wall separating a left atrium and a coronary sinus. The sensor implant device can comprise a first sensor component 1105 to obtain measurements at the left atrium and/or a second sensor component 1105 to obtain measurements at the coronary sinus.

In some examples, the sensor device 1102 and/or sensor component 1105 may form a generally cylindrical and/or tubular form having generally linear/straight sides. However, the sensor device 1102 may have an uneven surface and/or may comprise one or more pegs and/or portions having an increased diameter with respect to other portions of the sensor device 1102. For example, the sensor component 1105 may have an increased diameter compared to other portions of the sensor device 1102. In this way, the sensor component 1105 may advantageously be prevented from becoming embedded in a tissue wall.

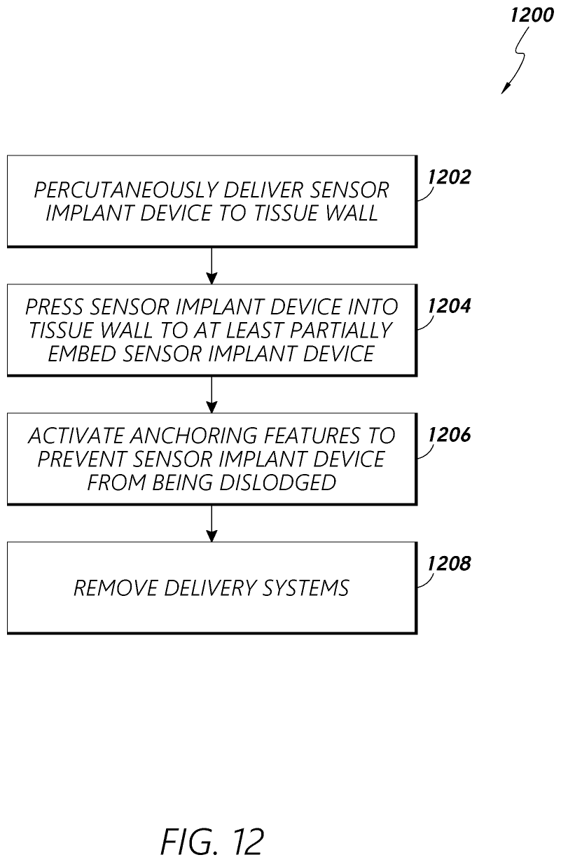

FIG. 12 provides a flowchart for an example process 1200 for percutaneous delivery and/or use of one or more of the various sensor implant devices described herein. Steps of the process 1200 may be performed in any order and/or may be repeated. For example, while the process 1200 described delivery of only a single sensor implant devices, multiple sensor implant devices may be delivered via one or more delivery systems.

At step 1202, the process 1200 involves percutaneously delivering a sensor implant device to a target tissue wall of a heart. The tissue wall may be, for example, a wall of the left atrium separating the left atrium from the coronary sinus. In this example, the sensor implant device may be delivered to a left atrium side of the tissue wall and/or to a coronary sinus side of the tissue wall. For example, the sensor implant device may be delivered via an atrial septal wall between the left atrium and the right atrium and/or via the coronary sinus and through an opening in the tissue wall to enter the left atrium. In another example, the sensor implant device may be delivered via the coronary sinus to the coronary sinus side of the tissue wall.

The sensor implant device can comprise a sensor body/device and/or one or more anchoring features. The sensor device can comprise at least one sensor component configured to obtain measurements related to blood flow characteristics at or near the sensor implant device. In some examples, the sensor device can comprise a first sensor component at or near a first end of the sensor device and/or can comprise a second sensor component at or near a second end of the sensor device. For example, the sensor implant device may be configured such that the first end of the sensor device extends into and/or adjacent to a first heart chamber and/or blood flow pathway (e.g., the left atrium) and/or the second end of the sensor device extends into and/or adjacent to a second heart chamber and/or blood flow pathway (e.g., the coronary sinus).

In some examples, the sensor implant device may be delivered via a catheter and/or shaft. The sensor implant device may comprise anchoring features configured to expand and/or be expanded following removal from the catheter and/or shaft. In some examples, the sensor implant device may be configured to assume a compressed and/or unexpanded form while within the catheter and/or shaft, which may advantageously minimize the delivery profile of the catheter, shaft and/or sensor implant device. In some examples, the catheter and/or shaft may comprise a pointed tip and/or other features configured to facilitate puncturing and/or advancing the catheter, shaft, and/or sensor implant device into the tissue wall.

At step 1204, the process 1200 involves pressing the sensor implant into the tissue wall to at least partially embed the sensor implant device within the tissue wall. In some examples, the sensor implant device may comprise a pointed tip and/or other features configured to facilitate puncturing and/or advancing the sensor implant device into the tissue wall. For example, a pointed tip of the sensor implant device may extend out of a catheter to allow the pointed tip of the sensor implant device to contact and/or pierce a surface of the tissue wall.

In some examples, the sensor implant device may be configured such that a physician may push the sensor implant device into the tissue wall. For example, one or more pushers and/or similar devices may be extended behind the sensor implant device within a catheter and/or sheath and/or may be utilized to press the sensor implant device out of the catheter and/or sheath and/or into contact with the tissue wall.

At step 1206, the process 1200 involves activating and/or engaging one or more anchoring features of the sensor implant device to anchor the sensor implant device within the tissue wall and/or to prevent the sensor implant device from being dislodged from the tissue wall. In some examples, the one or more anchoring features can comprise arms and/or hooks configured to extend from the sensor implant device and/or to form an approximately 45° angle with the sensor device.

In some examples, the one or more anchoring features may be configured to be situated against and/or within the sensor device of the sensor implant device during delivery to and/or into the tissue wall. The one or more anchoring features may be configured to extend away from the sensor device manually and/or naturally following removal from a catheter and/or other delivery systems. For example, one or more pull wires may be attached to the one or more anchoring features and/or may be configured to activate the one or more anchoring features by pulling the one or more anchoring features away from the sensor device. In another example, the one or more anchoring features may be configured to expand naturally while within the tissue wall.

The one or more anchoring features may be angled such that the one or more anchoring features may not restrict advancement of the sensor implant device through the tissue wall. For example, the one or more anchoring features may be configured to expand and/or extend in the direction of advancement through the tissue wall. Thus, as the sensor implant device is pressed into the tissue wall, the movement of the sensor implant device may cause the one or more anchoring features to be pressed against and/or within the sensor implant device. In some examples, the one or more anchoring features may be configured to extend to an approximately 135° angle with respect to a leading end of the sensor implant device and/or to an approximately 45° angle with respect to a tail end of the sensor implant device. After the sensor implant device has been advance through the tissue wall, the sensor implant device may be at least partially retracted into the tissue wall to cause activation and/or expansion of the one or more anchoring features. For example, a direction of retraction of the sensor implant device may be opposite a direction of expansion of the one or more anchoring features, such that retracting the sensor implant device may cause a force against the one or more anchoring features that may pull the one or more anchoring features away from the sensor device.

At step 1208, the process 1200 may involve removing one or more catheters, sheaths, pushers, pull wires, and/or other delivery systems while leaving the sensor implant device embedded within the tissue wall.

Some implementations of the present disclosure relate to a sensor implant device comprising a sensor body comprising, a first sensor component, and one or more anchoring features coupled to the sensor device and configured to anchor within a tissue wall. The one or more anchoring features are configured to assume an unexpanded form during delivery and configured to expand to anchor into the tissue wall.

The one or more anchoring features are configured to lay flatly against a surface of the sensor body in the unexpanded form. In some examples, the sensor body comprises one or more receptors. The one or more anchoring features may be configured to enter the one or more receptors in the unexpanded form.

In some examples, the one or more receptors are situated at an end portion of the sensor body. The end portion may have a conical shape.

The end portion may have a pointed shape. In some examples, each of the receptors comprises one or more springs.

In some examples, the one or more receptors comprise indentations in the sensor body. The one or more anchoring features may couple to the sensor device via hinge joints.

The one or more anchoring features may be configured to expand to an approximately 45° angle with respect to a surface of the sensor body in the expanded form. In some examples, the one or more anchoring features are configured to expand to no more than a 90° angle with respect to a surface of the sensor body in the expanded form.

In some examples, the one or more anchoring features are configured to swing freely between the unexpanded form and the expanded form. The one or more anchoring features may be biased in the expanded form.

The one or more anchoring features may be spring-loaded. In some examples, the sensor body comprises a pointed tip configured to pierce the tissue wall.

In some examples, the pointed tip is at a conical end portion of the sensor body. The one or more anchoring features may be coupled to the conical end portion of the sensor body.

The first sensor component may have a greater width than the sensor body. In some examples, the first sensor component is situated at a first end of the sensor body.

The one or more anchoring features may extend from a second end of the sensor body. In some examples, the sensor implant device further comprises a second sensor component situated at a second end of the sensor body.

In some examples, the one or more anchoring features extend from a midsection of the sensor body. The one or more anchoring features may comprise pointed arms.

The one or more anchoring features may extend, in the unexpanded form, towards the first sensor component. In some examples, the one or more anchoring features extend, in the unexpanded form, away from the first sensor component.

In some examples, the one or more anchoring features comprise four anchoring features. The one or more anchoring features may comprise eight anchoring features.

In accordance with some implementations of the present disclosure, a method comprises percutaneously delivering a sensor implant device within a catheter to a tissue wall. The sensor implant device comprises one or more anchoring features configured to assume a compressed form while within the catheter. The method further comprises piercing the tissue wall to embed the sensor implant device at least partially within the tissue wall and removing the sensor implant device from the catheter. The one or more anchoring features are configured to assume an expanded form following removal from the catheter.

The catheter may comprise a pointed tip. In some examples, piercing the tissue wall is performed using the pointed tip of the catheter.

In some examples, the sensor implant device comprises a pointed tip. Piercing the tissue wall may be performed using the pointed tip of the sensor implant device.

The one or more anchoring features may be configured to lay flatly against a surface of the sensor implant device in the compressed form. In some examples, the sensor implant device comprises one or more receptors. The one or more anchoring features may be configured to enter the one or more receptors in the compressed form.

In some examples, the one or more anchoring features are configured to swing freely between the compressed form and the expanded form. one or more anchoring features may be biased in the expanded form.

### ADDITIONAL EXAMPLES

Depending on the example, certain acts, events, or functions of any of the processes or algorithms described herein can be performed in a different sequence, may be added, merged, or left out altogether. Thus, in certain examples, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain examples require at least one of X, at least one of Y and at least one of Z to each be present.

It should be appreciated that in the above description of examples, various features are sometimes grouped together in a single example, figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the inventions herein disclosed and claimed below should not be limited by the particular examples described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example examples belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A sensor implant device comprising:
a sensor body comprising;
a first sensor component; and
one or more anchoring features coupled to the sensor body and configured to anchor within a tissue wall, the one or more anchoring features configured to assume an unexpanded form during delivery and configured to expand to anchor into the tissue wall.

2. The sensor implant device of claim 1, wherein the sensor body comprises one or more receptors, and wherein the one or more anchoring features are configured to enter the one or more receptors in the unexpanded form.

3. The sensor implant device of claim 2, wherein the one or more receptors are situated at an end portion of the sensor body.

4. The sensor implant device of claim 3, wherein each of the receptors comprises one or more springs.

5. The sensor implant device of claim 1, wherein the one or more anchoring features couple to the sensor body via hinge joints.

6. The sensor implant device of claim 1, wherein the one or more anchoring features are biased in an expanded form.

7. The sensor implant device of claim 6, wherein the one or more anchoring features are spring-loaded.

8. The sensor implant device of claim 1, wherein the sensor body comprises a pointed tip configured to pierce the tissue wall.

9. The sensor implant device of claim 8, wherein the pointed tip is at a conical end portion of the sensor body.

10. The sensor implant device of claim 9, wherein the one or more anchoring features are coupled to the conical end portion of the sensor body.

11. The sensor implant device of claim 1, wherein the first sensor component is situated at a first end of the sensor body.

12. The sensor implant device of claim 11, wherein the one or more anchoring features extend from a second end of the sensor body.

13. The sensor implant device of claim 1, wherein the one or more anchoring features extend from a midsection of the sensor body.

14. A method comprising:
percutaneously delivering a sensor implant device within a catheter to a tissue wall, the sensor implant device comprising one or more anchoring features configured to assume a compressed form while within the catheter;
piercing the tissue wall to embed the sensor implant device at least partially within the tissue wall; and
removing the sensor implant device from the catheter, wherein the one or more anchoring features are configured to assume an expanded form following removal from the catheter.

15. The method of claim 14, wherein the catheter comprises a pointed tip, and piercing the tissue wall is performed using the pointed tip of the catheter.

16. The method of claim 14, wherein the sensor implant device comprises a pointed tip, and wherein piercing the tissue wall is performed using the pointed tip of the sensor implant device.

17. The method of claim 14, wherein the one or more anchoring features are configured to lay flatly against a surface of the sensor implant device in the compressed form.

18. The method of claim 14, wherein the sensor implant device comprises one or more receptors, and wherein the one or more anchoring features are configured to enter the one or more receptors in the compressed form.

19. The method of claim 14, wherein the one or more anchoring features are configured to swing freely between the compressed form and the expanded form.

20. The method of claim 14, wherein the one or more anchoring features are biased in the expanded form.
